(19) United States
(12) Patent Application Publication
(10) Pub. No.: US 2011/0144539 A1
(43) Pub. Date: Jun. 16, 2011

(54) Concussion Warning Apparatus

(76) Inventor: Norman Ken Ouchi, San Jose, CA (US)

(21) Appl. No.: 12/636,700

(22) Filed: Dec. 11, 2009

(51) Int. Cl. A61B 5/103

(52) U.S. Cl. 600/587

(57) ABSTRACT

The present invention is related to detecting possible concussions due to blunt force blows or shocks to the head. In the present invention, areas of the athlete's head are covered with a blunt force blow indicating sheet prior to the possible concussion causing activity and the sheet is examined for indications of blunt force blows that may have caused a concussion. The athlete with indications of possible concussion is then examined to determine if the athlete suffered a concussion. The blunt force blow indicating sheet may be used to line a helmet or headgear used by athletes or the sheet may be shaped like a helmet for use in activities where headgear is not worn. Objects that may be subject to blunt force blows, during for instance shipping, is covered with the sheet to detect if the object experienced a blunt force blow that may have damaged the object.

### CROSS-REFERENCE TO RELATED APPLICATIONS

None

### STATEMENT REGARDING FEDERALLY SPONSORED RESEARCH OR DEVELOPMENT

None

### FIELD OF THE INVENTION

The present invention is related to detecting possible concussions due to blunt force blows or impacts to the head.

### BRIEF SUMMARY OF THE INVENTION

In the present invention, areas of the athlete's head are covered with a blunt force blow indicating sheet prior to the possible concussion causing activity and the sheet is examined for indications of blunt force blows that may have caused a concussion. The athlete with indications of possible concussion is then examined to determine if the athlete suffered a concussion. The blunt force blow indicating sheet may be used to line a helmet or headgear used by athletes or the sheet may be shaped like a helmet for use in activities where headgear is not worn. An object that may be subject to blunt force blows, for instance during shipping, is covered with the sheet to detect if the object experienced a blunt force blow that may have damaged the object.

### BACKGROUND OF THE INVENTION

A concussion is a bruising of the brain caused by a blunt force blow that causes the brain to slam within the skull as illustrated in FIG. 1. The blow may be directly to the head or may be the result of the head being hit by the inside of a helmet or headgear as the helmet takes a significant part of the blow but transmits some of the blow through the cushioning material of the helmet as illustrated in FIG. 2 where the larger external blunt force blow is transformed into a smaller blunt force blow by the cushion layer of the helmet. Professional and high school athletes suffer head injuries/concussions every day. Since signs, symptoms, and after effects can be subtle and sometimes undetected by coaches, trainer, parents and medical providers, it is called the Invisible Injury. This makes it even more important for coaches, trainers, Emergency Rooms, doctors, and parents to use due diligence in examining the athletes and conducting necessary medical testing and evaluation to help diagnose the injury.

Concussion detection is a major problem. Most people responsibe for the athlete are not trained to detect a concussion since it requires evaluation of the player's responses and is very subjective. McCrea, Kelly, and Randolph provide a Standardized Assessment of Concussion (SAC) Manual, standardized forms, and pock guides in an attempt to bring objectivity to the concussion assessment. Even with standardization of the evaluation process, concussions cannot be detected until there are visible behavioral changes in the athlete. Many concussions go undetected because the blows are accumulated over time and the impairment becomes visible long after the accumulated blows could have been prevented.

Many activities that cause concussion are recognized and participants wear protective helmets or headgear to absorb the impact of the blow. Athletes participating in football, hockey, and other contact sports wear helmets. Children and adults who ride bikes, skate, skateboard, ski, snowboard or other activities also wear helmets. Some games, such as soccer, where participants do not wear headgear may cause concussions where participants collide or strike the ball with their heads. Baseball players wear batting helmets while at bat but only caps while in the field. Players may collide in play or hit the fence catching a long fly ball and suffer blows to the head that may cause concussions.

Desired is an apparatus that can detect possible concussions independent of the assessment by athlete or behavior of the athlete so that when detected, the athlete can undergo further evaluation.

The apparatus should provide:

little or no interference to the athlete;

visible or other indication so that little training is required to determine if a possible concussion is detected;

use in real time by the athlete during practices and games;

as an addition but not necessary, indication of accumulated trauma that over time may lead to a concussion;

### BRIEF DESCRIPTION OF DRAWINGS

FIG. 1 illustrates a schematic cross section of the brain, skull, and a blunt force blow to the skull that may cause a concussion.

FIG. 2 illustrates a schematic cross section of the brain, skull, cushion, helmet, and a blunt force blow that is absorbed and attenuated by the cushion with a resulting smaller blunt force blow to the skull that may cause a concussion.

FIG. 3 is a photo of a section of bubble wrap with its array of air encapsulated bubbles.

FIG. 4 illustrates a schematic cross section of the brain, skull, indicator sheet 1, cushion, helmet, and a blunt force blow that is absorbed and attenuated by the cushion with a resulting smaller blunt force blow to indicator sheet 1 and the skull that may cause a concussion.

FIG. 5 illustrates a schematic cross section of the brain, skull, indicator sheet 1, and a blunt force blow to indicator sheet 1 and the skull that may cause a concussion.

FIG. 6A illustrates a section of an indicator sheet 1 comprised of encapsulated air, gas, liquid, or powder where encapsulations burst with a blunt force blow above a specified impact.

FIG. 6B illustrates a section of the indicator sheet 1 of FIG. 6A where six encapsulations have burst indicated in gray due to a blunt force greater than the specified impact providing a visual indication of a blunt force blow in that area that may cause a concussion.

FIG. 7A illustrates a section of an indicator sheet 1 comprised of filaments where filaments break with a blunt force blow of specified impact.

FIG. 7B illustrates a section of the indicator sheet 1 of FIG. 7A where four filaments have broken due to a blunt force greater than the specified impact providing a visual indication of a blunt force blow in that area that may cause a concussion.

FIG. 8A illustrates a section of an indicator sheet 1 comprised of ampoules of air, gas, liquid, or powder where ampoules burst with a blunt force blow of specified impact.

FIG. 8B illustrates a section of the indicator sheet 1 of FIG. 8A where three ampoules have burst indicated in gray due to a blunt force greater than the specified impact providing a visual indication of a blunt force blow in that area that may cause a concussion.

FIG. 9 illustrates a schematic cross section of the brain, skull, crush cushion 2, helmet, and a blunt force blow to the helmet, crush cushion 2, and the skull that may cause a concussion.

FIG. 10A illustrates crush cushion 2 before a blunt force blow that may cause a concussion.

FIG. 10B illustrates a deformed crush cushion 2 after a blunt force blow that may cause a concussion.

FIG. 11A illustrates a cross section of the indicator sheet 1 with indicators of two sizes where the larger indicator bursts with first lower impact and the smaller indicator burst with a second lower impact to the same area after the larger adjoining indicators have burst. A single large impact bursts both large and small indicators.

FIG. 11B illustrates a cross section of the indicator sheet 1 after a first lower impact bursting larger indicators at the area of the blunt force blow.

FIG. 11C illustrates a cross section of the indicator sheet 1 after a second lower impact bursting smaller indicators at the area of the second blunt force blow or after a first higher impact bursting large indicators and small indicators at the area of the first higher blunt force blow.

FIG. 12A illustrates a cross section of the indicator sheet 1 with indicators comprising two layers where the outer indicator bursts with first lower impact and the inner indicator burst with a second lower impact to the same area after the outer indicators have burst. A single large impact bursts both outer and inner indicators.

FIG. 12B illustrates a cross section of the indicator sheet 1 after a first lower impact bursting the outer indicators at the area of the blunt force blow.

FIG. 12C illustrates a cross section of the indicator sheet 1 after a second lower impact bursting inner indicators at the area of the second blunt force blow or after a first higher impact bursting both outer and inner indicators at the area of the first higher blunt force blow.

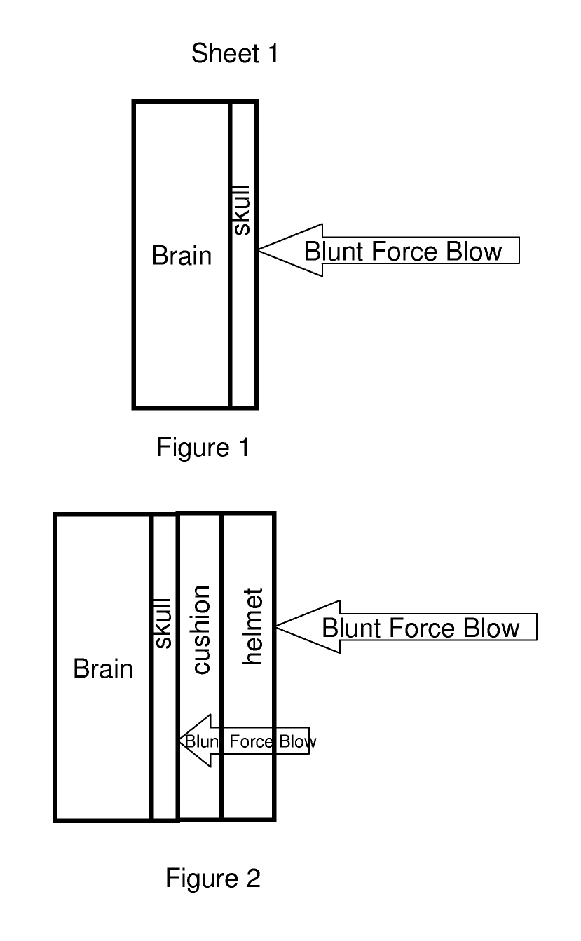

### DESCRIPTION OF THE INVENTION

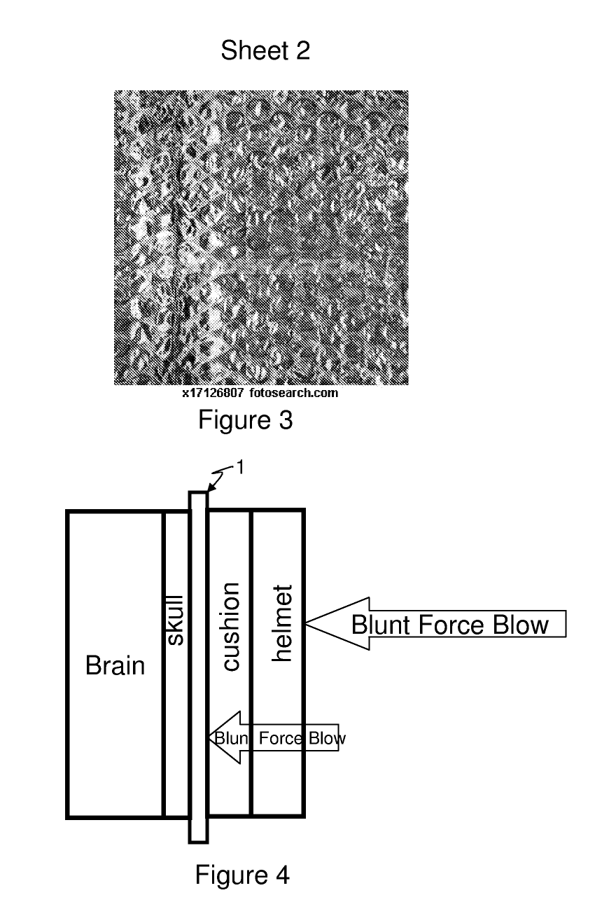

A concussion is caused by a blunt force blow to the head that causes physical damage to the brain usually in the form of burst blood capillaries. The present invention provides warning of possible concussion by indicating when a blunt force blow with impact or force sufficient to cause a concussion has been experienced by the head. A concussion is a hidden bruising of the brain and the present invention makes visible a potential bruising blunt force blow to the head by a “bruise” to a layer or sheet covering areas of the head subject to the blunt force blow. The present invention provides an indication of the location of the blow and can provide a sense of the intensity or shock of the force. The blunt force blow indicator 1 is a sheet where an area of the sheet changes physically when that area is subject to a blunt force blow greater than a specified impact. A sheet of bubble wrap used to cushion objects for shipping of FIG. 3 is an illustrative example of a blunt force blow indicator where air is encapsulated in bubbles between two sheets of flexible plastic. An encapsulated bubble can be popped, changed, by a blunt force blow as can be attested by many who have popped bubble wrap. The encapsulated bubbles in bubble wrap are not designed to pop at a specified blunt force impact but can be designed as an indicator to pop when an impact greater than a specified impact or shock is applied. The areas of the athlete's head subject to concussive blunt force blows are covered with the indicator sheet 1. For activities where helmets or headgear is worn, the interior of a helmet or headgear is lined with the indicator sheet 1 material so the athlete is not impaired by the application of the indicator sheet material as illustrated in FIG. 4. The indicator sheet 1 is checked after a blunt force hit to the athlete or periodically for indication that a blunt force impact has popped—changed—any of the bubbles. A popped bubble is a warning of possible concussion and the athlete should be checked for a concussion. FIG. 6A illustrates an array of bubbles before a blunt force impact. FIG. 6B illustrates in gray, six bubbles that have popped and indicative of a blunt force blow with impact great enough to pop the bubbles. The gray area provides visual indication of the location of the blow; the invisible potential bruising of the brain is made visible by the indicator sheet.

The bubbles may be filled with a dye or other liquid and be small compared to the bulk of the indicator sheet 1 so that the bubbles are not noticeable. When the bubbles burst releasing the dye, the indication spreads across an area of the indicator sheet 1 making visible the area of the blunt force blow much as the blood in burst capillaries makes visible a bruise of an injury due to a blunt force blow.

The indicator sheet 1 provides indication that is easily identifiable and easy to train coaches, trainer, parents, medical providers and athletes to watch for these concussion warnings.

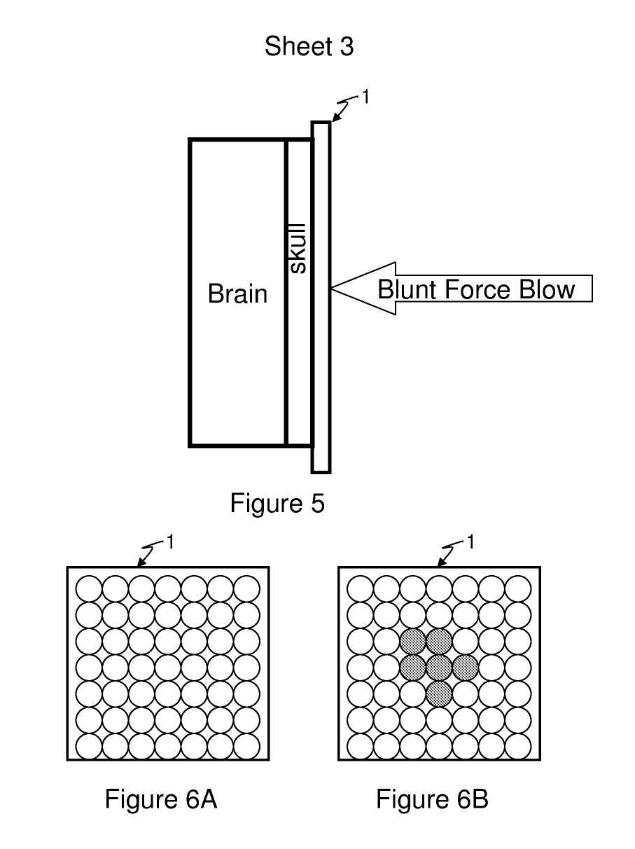

The indicator sheet 1 can line baseball caps and other headgear that are not designed to protect against head blows and provide a concussion warning without significant impact to the athlete wearing the cap or headgear as illustrated in FIG. 5.

The indicator sheet 1 may be light weight . . . like bubble wrap . . . and when shaped and worn as a very light weight helmet, athletes playing games where headgear is not normally worn, such as soccer, can wear concussion warning headgear as illustrated in FIG. 5. The indicator sheet 1 may be perforated for ventilation to reduce the impact to the athlete.

The human head is not uniformly subjected to concussion and specific areas such as the sides, back, and front of the head may suffer a concussion with lower impact blows than the top of the head. The indicator sheet 1 may be designed where lower impact blows to these areas to indicate a blow of possible concussive force while a higher impact blow is required to indicate a blow to the higher impact blow area.

The indicator sheet may be designed to indicate the range of impact of the blow where a first set of indicators trigger at a first impact and a second set of indicators trigger at a second, higher, impact such that the impact of a blow can be estimated to be greater than the first impact but less than the second impact or greater than the second impact.

Similarly, the size of the impact indicators may be of smaller size in areas of the head where more sensitivity to the impact is required or where the contours of the head require smaller indicator sizes for coverage and more accurate location of the blow.

Concussions can be caused by multiple blows of lower impact. The blows may be over a period of time. The impact indicator may be designed such that a number of blows of lower impact indicate a possible concussion or indicates immediately if a higher impact blow is experienced. The indicators are designed where a number of units are activated in sequence by lower impact blows where that number of lower impact blows are required to trigger the indicator while a single higher impact blow activates two or more of the units in the sequence and may with one blow trigger the indicator.

With dye filled bubbles, a large blow would leave a larger “bruise”. A small blow would result in a small “bruise” that is not noticeable. However, multiple small blows result in a larger “bruise” that is noticed. This provides means to detect consecutive small blows to an area and warn of a possible concussion.

The impact indicator provides visible indication that a blow of a specified impact or larger has been experienced in that area of the sheet, e.g. a popped bubble in bubble wrap. The indicator may include a dye that is released or other visible physical change when impacted by a blow.

The impact indicators may be integrated with cushioning material in a helmet or headgear where the impact indicator may provide some cushioning for lower impacts but still change to indicate a blow when a blow greater than the specified force is experienced. A cushion material may rebound from a blow lower than a specified impact but remain deformed after a blow of greater impact as illustrated in FIG. 10A where the cushion material aids in absorbing the blunt force impact below a specified force without deformation but deforms as illustrated in FIG. 10B when a higher force impact is absorbed.

The impact indicator material may be used to cover objects other than heads where indications of impacts are required. An object during shipping may experience impacts that create hidden damage to the object and the indicator material provides a warning when the object experienced impacts greater than a specified force.

### DESCRIPTION OF A PREFERRED EMBODIMENT

Blunt force blow indicator sheet 1 may be constructed in many ways and several embodiments are disclosed.

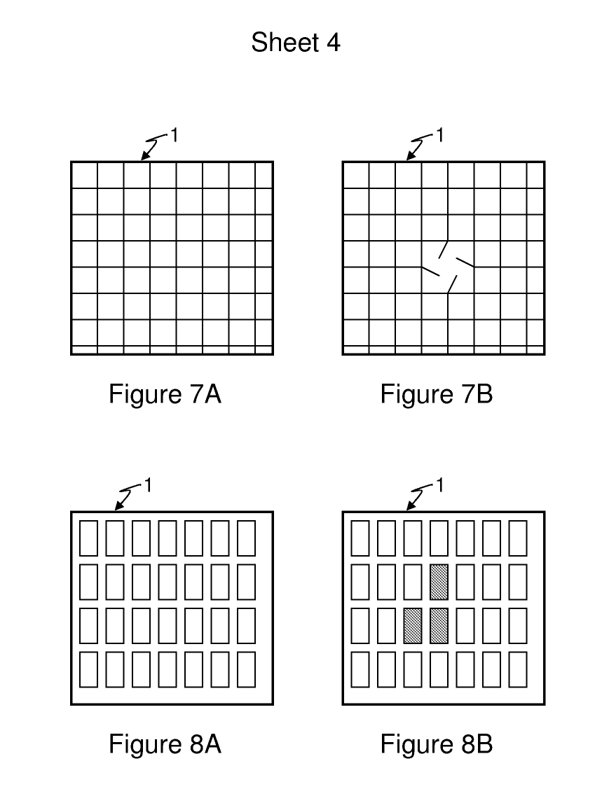

The bubble wrap like material is one of many possible blunt force blow indicators where material is altered, changed, by a blunt force blow greater than a specified impact. Bubble wrap encapsulates air; an indicator sheet 1 may encapsulate air, gases, fluids, powders, or any substance that will indicate when a blunt force blow has burst an encapsulation. Another example is small hollow balls of plastic may be made to burst at a specified impact or force and sandwiched between sheets. The bubble wrap or hollow balls are illustrated in FIG. 6A with all indicators intact and FIG. 6B with six indicators changed and shaded in gray. A third example is fibers that break at a specified tension woven in a mesh and sandwiched between sheets of plastic where an impact to the sandwiched mesh causes fibers to break as an indication of an impact greater than the specified impact. These are illustrated in FIG. 7A with all fibers intact and FIG. 7B with four fibers broken. A fourth example is small ampoules of dye sandwiched between plastic sheets where an ampoule is designed to fracture at a specified impact and the dye is released between the sheets. These are illustrated in FIG. 8A with all ampoules intact and FIG. 8B with three ampoules fractured.

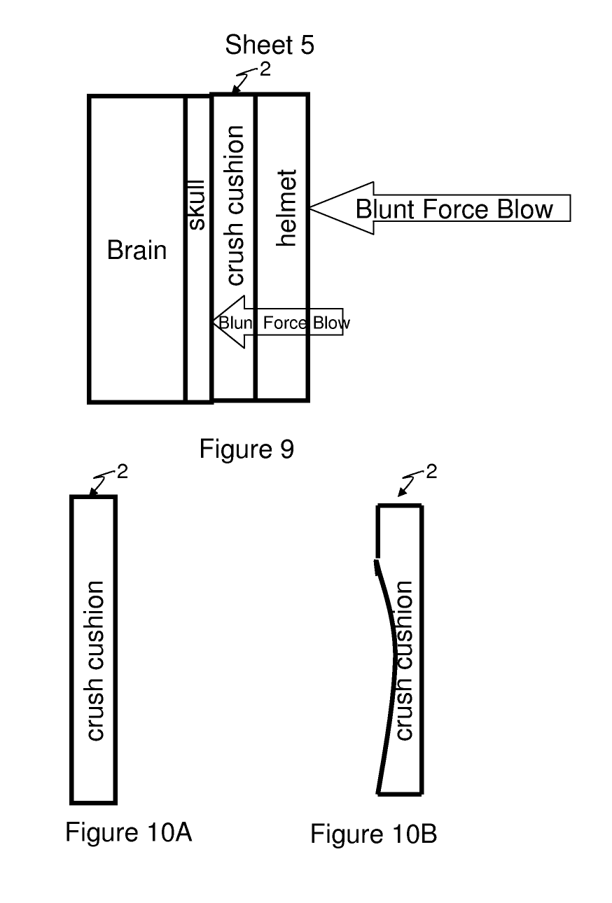

A fifth example is flexible, compressible foam that deforms at a specified force or impact. The foam may be a crush cushion 2 illustrated in FIG. 9 where the crush cushion 2 serves as the impact absorbing cushion for a helmet. The crush cushion 2 maintains its form for blunt force blow impacts less than the specified impact as illustrated in FIG. 10A and deforms with higher impact as illustrated in FIG. 10B.

A sixth example is the integration of bubbles or hollow balls or ampoules into the cushion layer of a helmet where the encapsulated indicator bursts when a blunt force impact greater than the designed impact compresses the cushion layer as an indication of a possible concussion.

All of the indicators provide visual indication when a blunt force blow of impact greater than a specified value is experienced at the area of the indication. Those of ordinary skill may construct other materials that change physical structure to indicate a blunt force blow of greater than a specified impact has been experienced on an area of the material.

The blunt force blow indicators have a small thickness that is compressed by the blow, e.g. the bubbles in bubble wrap have thickness, and provide a measure of force absorption to lessen the blow. A blunt force blow indicator absorbs some of the energy of the blunt force blow to change the indicator and reduces the impact to the indicator sheet 1 and the head. This can be used to indicate cumulative blows to an area.

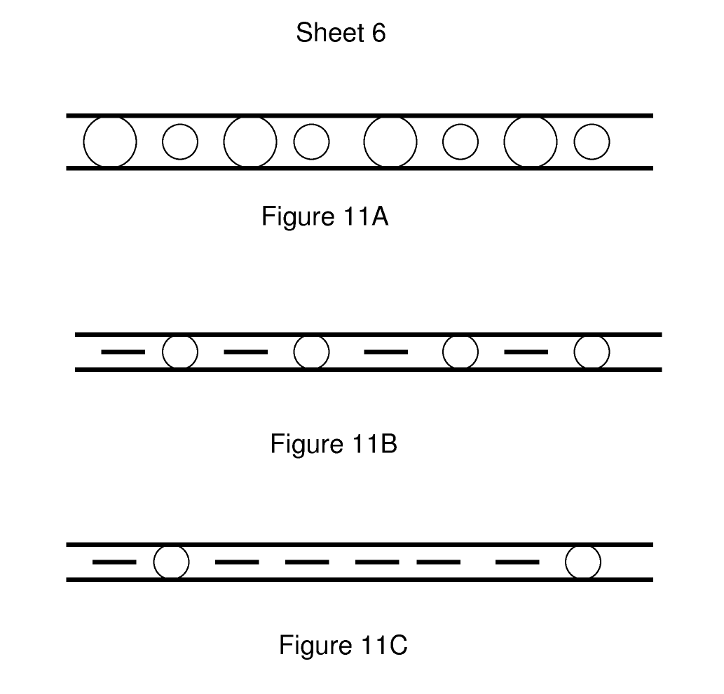

The indicator sheet 1 provides means to indicate two accumulated blows of lower magnitude impact where encapsulations that burst a the lower magnitude impact are made in two sizes and sandwiched between two flexible sheets as illustrated in FIG. 11A. A first lower magnitude impact bursts the larger encapsulations that absorbs the energy of the impact leaving the smaller encapsulations intact as illustrated in FIG. 11B. A second lower magnitude impact burst two of the remaining smaller encapsulations indicating a second impact was experienced as illustrated in FIG. 11C. A single higher magnitude impact bursts both large and small encapsulations resulting in a similar indication as two lower magnitude impacts illustrated in FIG. 11C.

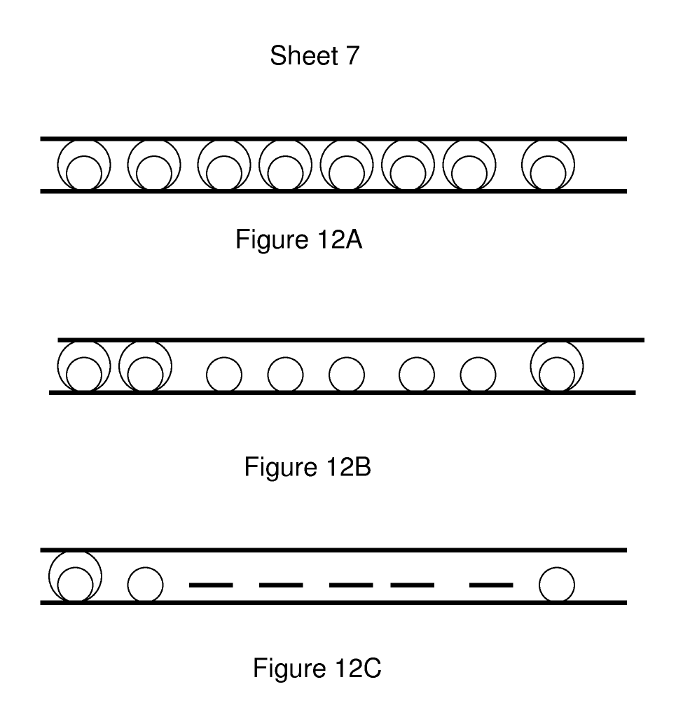

A second indicator sheet 1 provides means to indicate two accumulated blows of lower magnitude impact where encapsulations that burst a the lower magnitude impact are made in two sizes, the smaller size is packaged within the larger size and the double layered indicators are sandwiched between two flexible sheets as illustrated in FIG. 12A. A first lower magnitude impact bursts the larger encapsulations that absorb the energy of the impact leaving the smaller encapsulations as illustrated in FIG. 12B. A second lower magnitude impact burst five of the remaining smaller encapsulations indicating a second impact was experienced as illustrated in FIG. 12C. A single higher magnitude impact bursts both large and small encapsulations resulting in a similar indication as two lower magnitude impacts illustrated in FIG. 12C.

Indicator encapsulations that indicate three accumulated blows of lower magnitude impact may be devised by requiring three lower impact blows in sequence before indicating a possible concussion while indicating when a single high impact blow has been experienced. Those of ordinary skill may construct other materials that change physical structure to indicate accumulated blunt force blows of greater than a specified impact but less than an impact that might cause a concussion or a single blow with impact that might cause a concussion has been experienced on an area of the material.

I claim:

1. A concussion warning apparatus comprising:
an indicator sheet with indicators where an indicator changes when struck with a blunt force blow impact that is sufficient to cause a concussion;
areas of an athlete's head are covered by the indicator sheet prior to an activity that may cause a concussion;
the indicator sheet is examined for a changed indicator as a warning of a blunt force blow that may have caused a concussion.

2. The indicator sheet of claim 1 is used to line the inside of a helmet or headgear used by the athlete.

3. The indicator sheet of claim 1 provides a first indicator that changes at first blunt force blow impact and a second indicator that changes at a second blunt force blow impact different from the first blunt force blow impact.

4. The indicator sheet of claim 1 provides indicators that change at different blunt force blow impact and the areas of the athlete's head that are subject to different blunt force blow impacts that may cause a concussion are covered with indicators with change force impact appropriate for the area.

5. The indicator sheet of claim 1 provides indicators that change after two blunt force blows that are of lower impact where a single blow of lower impact would not change the indicator but the indicator would change with a single blow of higher impact.

6. The indicator sheet of claim 1 where a changed indicator provides a color change indication that spreads across an area of the indicator sheet.

7. The indicator sheet of claim 1 provides a first indicator of a first size and a second indicator of a second size different from the first size.

8. The indicator sheet of claim 1 provides indicators of different sizes and the areas of the athlete's head that require higher resolution indication of the impact area are covered with smaller size indicators than areas with lower resolution indication with larger size indicators.

9. The indicator sheet of claim 1 is shaped to conform to an athlete's head and worn by the athlete without a helmet or headgear.

10. The indicator sheet of claim 1 where the indicators are integrated with the cushion layer of a helmet and the cushion layer is examined for a blunt force blow indication as a warning of a possible concussion.

11. A blunt force blow indicating sheet for warning of concussions where areas of the head are covered by the indicating sheet prior to an activity that may cause concussions and the sheet is examined for a blunt force blow indication as a warning of a possible concussion.

12. The indicating sheet of claim 11 is used to line a helmet or headgear.

13. The indicating sheet of claim 11 where the sheet provides a visual indicator that spreads across the area where a blunt force has been experienced.

14. The indicating sheet of claim 11 where the sheet provides an indication of two blunt force blows of lower intensity to an area of the sheet where a single blow of lower intensity would not have been sufficient for an indication but a single blow of higher intensity is sufficient for an indication.

15. The indicating sheet of claim 11 where the sheet is shaped like a helmet and worn without a helmet.

16. The indicating sheet of claim 11 where the indicators are integrated with the cushion layer of a helmet and the cushion layer is examined for a blunt force blow indication as a warning of a possible concussion.

17. A blunt force blow indication sheet covering an object where the sheet indicates a blunt force blow that may have caused damage to the object.

18. The indication sheet of claim 17 covering areas of an athlete's head subject to concussion prior to activities that may cause a concussion and the indication sheet is checked for blunt force blows that may have caused a concussion.

19. The indication sheet of claim 17 is used to line the inside of a helmet or headgear used by an athlete and checked for blunt force blows that may have caused a concussion.

20. The indication sheet of claim 17 wherein the indicators of the sheet are incorporated into the blunt force cushioning material around an object such that the indicators indicate a blunt force blow that may cause damage to the object was experienced by the cushioning material.
